(19) United States
(12) Patent Application Publication
(10) Pub. No.: US 2023/0248926 A1
(43) Pub. Date: Aug. 10, 2023

(54) AUTOMATED OXYGEN MONITORING SYSTEMS TO TITRATE SUPPLEMENTAL OXYGEN, ARRANGEMENTS INCORPORATING THE SAME, AND METHODS THEREFOR

(71) Applicant: Neal Ramchandani, Indianapolis, IN (US)

(72) Inventor: Neal Ramchandani, Indianapolis, IN (US)

(21) Appl. No.: 17/665,767

(22) Filed: Feb. 7, 2022

(51) Int. Cl. A61M 16/00; A61M 16/10; A61B 5/145; A61M 16/20

(52) U.S. Cl. CPC A61M 16/0003; A61M 16/1005; A61B 5/14542; A61M 16/201

(57) ABSTRACT

Arrangements, systems, and methods to deliver supplemental oxygen to a patient are disclosed. An arrangement to deliver supplemental oxygen to a patient includes a source of supplemental oxygen, an outlet station, a sensor, and an automated oxygen monitoring system. The outlet station includes at least one valve fluidly coupled to the supplemental oxygen source and configured to permit flow of supplemental oxygen therethrough. The sensor is directly attachable to the patient and configured to provide a signal indicative of a measured blood oxygen saturation level of the patient. The automated oxygen monitoring system communicatively coupled to the sensor and in fluid communication with a tube adapted for administration of supplemental oxygen to the patient.

### FIELD OF THE DISCLOSURE

The present disclosure relates, generally, to oxygen therapy, and, more specifically, to systems for delivering supplemental oxygen to patients.

### BACKGROUND

Current systems and/or devices adapted to deliver supplemental oxygen to patients may suffer from a variety of drawbacks. In some applications, such systems and/or devices may provide, or otherwise be associated with, limited performance, undesirable cost, or excessive complexity. Apparatuses and/or methods that avoid the aforementioned shortcomings remain an area of interest.

### SUMMARY

The present disclosure may comprise one or more of the following features and combinations thereof.

According to one aspect of the present disclosure, an arrangement to deliver supplemental oxygen to a patient may include a source of supplemental oxygen, an outlet station, a sensor, and an automated oxygen monitoring system. The outlet station may include at least one valve fluidly coupled to the supplemental oxygen source and configured to permit flow of supplemental oxygen therethrough. The sensor may be directly attachable to the patient and configured to provide a signal indicative of a measured blood oxygen saturation level of the patient. The automated oxygen monitoring system may be communicatively coupled to the sensor and in fluid communication with a tube adapted for administration of supplemental oxygen to the patient. The oxygen monitoring system may be in fluid communication with the source of supplemental oxygen to receive the flow of supplemental oxygen through the at least one valve. The oxygen monitoring system may include a controller having a memory device and a processor coupled thereto. The memory device may have instructions stored therein that are executable by the processor to cause the processor to receive the signal from the sensor, to titrate supplemental oxygen for delivery to the patient through the tube based on the signal, and to continuously monitor the blood oxygen saturation level of the patient based on the signal.

In some embodiments, the arrangement may include an adaptor communicatively coupled to the sensor and the automated oxygen monitoring system, and the adaptor may be configured to transmit the signal provided by the sensor to the processor. The arrangement may include an oxygen saturation monitor communicatively coupled to the adaptor, and the adaptor may be configured to transmit the signal provided by the sensor to the oxygen saturation monitor to permit continuous measurement of the blood oxygen saturation level of the patient via the oxygen saturation monitor. The oxygen saturation monitor may be provided separately from the automated oxygen monitoring system. Additionally, in some embodiments, the automated oxygen monitoring system may include an override switch, and the override switch may be manually manipulatable by a user to selectively cease delivery of supplemental oxygen to the patient through the oxygen monitoring system.

In some embodiments, the instructions stored in the memory device may be executable by the processor to cause the processor to retrieve patient information specific to the patient and to titrate supplemental oxygen for delivery to the patient at an initial rate or an initial amount based on the signal and the patient information. The instructions stored in the memory device may be executable by the processor to cause the processor to compare the measured blood oxygen saturation level of the patient following delivery of supplemental oxygen to the patient at the initial rate or the initial amount to a reference pulse oximetric percentage, and to issue a warning in response to a determination that the measured blood oxygen saturation level of the patient does not meet the reference pulse oximetric percentage. The instructions stored in the memory device may be executable by the processor to cause the processor to titrate supplemental oxygen for delivery to the patient at an increased rate or an increased amount in response to the determination that the measured blood oxygen saturation level of the patient does not meet the reference pulse oximetric percentage. The instructions stored in the memory device may be executable by the processor to cause the processor to compare the measured blood oxygen saturation level of the patient following delivery of supplemental oxygen to the patient at the increased rate or the increased amount to the reference pulse oximetric percentage, and to issue an alarm in response to a determination that the measured blood oxygen saturation level of the patient does not meet the reference pulse oximetric percentage. The instructions stored in the memory device may be executable by the processor to cause the processor to, in response to a determination that the measured blood oxygen saturation level of the patient meets the reference pulse oximetric percentage, continue delivery of supplemental oxygen to the patient at the increased rate or the increased amount or cease further delivery of supplemental oxygen to the patient.

In some embodiments, the blood oxygen saturation level of the patient may be defined as a percentage of hemoglobin molecules bound by oxygen. Additionally, in some embodiments, the tube may be adapted for non-invasive administration of supplemental oxygen to the patient. Additionally, in some embodiments, the instructions stored in the memory may be executable by the processor to cause the processor to receive the signal from the sensor, to titrate supplemental oxygen for delivery to the patient through the tube based on the signal, and to continuously, or intermittently in a selective manner, monitor the supplemental oxygen of the patient based on the signal.

In some embodiments, the arrangement may include an adaptor communicatively coupled to the sensor and the automated oxygen monitoring system, and the adaptor may be configured to transmit the signal provided by the sensor to the processor. The arrangement may include a vital sign display unit having an oxygen saturation monitor communicatively coupled to the adaptor, and the adaptor may be configured to transmit the signal provided by the sensor directly to the oxygen saturation monitor to permit continuous measurement of the blood oxygen saturation level of the patient via the oxygen saturation monitor. The oxygen saturation monitor may be provided separately from the automated oxygen monitoring system. Additionally, in some embodiments, the automated oxygen monitoring system may include an override switch, and the override switch may be manually manipulatable by a user to selectively cease, increase, or decrease delivery of supplemental oxygen to the patient through the oxygen monitoring system.

In some embodiments, the instructions stored in the memory device may be executable by the processor to cause the processor to retrieve patient information specific to the patient and to titrate supplemental oxygen for delivery to the patient at an initial rate or an initial amount based on the signal and the patient information. The instructions stored in the memory device may be executable by the processor to cause the processor to compare the measured supplemental oxygen of the patient following delivery of supplemental oxygen to the patient at the initial rate or the initial amount to a reference pulse oximetric percentage, and to issue a warning in response to a determination that the measured supplemental oxygen of the patient does not meet the reference pulse oximetric percentage. The instructions stored in the memory device may be executable by the processor to cause the processor to titrate supplemental oxygen for delivery to the patient at an increased rate or an increased amount in response to the determination that the measured supplemental oxygen of the patient does not meet the reference pulse oximetric percentage. The instructions stored in the memory device may be executable by the processor to cause the processor to compare the measured supplemental oxygen of the patient following delivery of supplemental oxygen to the patient at the increased rate or the increased amount to the reference pulse oximetric percentage, and to issue an alarm in response to a determination that the measured supplemental oxygen of the patient does not meet the reference pulse oximetric percentage or a determination that supplemental oxygen has exceeded titration parameters. The instructions stored in the memory device may be executable by the processor to cause the processor to, in response to a determination that the measured blood oxygen saturation level of the patient meets the reference pulse oximetric percentage, continue delivery of supplemental oxygen to the patient at the increased rate or the increased amount or cease further delivery of supplemental oxygen to the patient.

According to another aspect of the present disclosure, an automated oxygen monitoring system to deliver supplemental oxygen to a patient may include a controller having a memory device and a processor coupled thereto. The memory device may have instructions stored therein that are executable by the processor to cause the processor to receive a signal from a sensor indicative of a measured blood oxygen saturation level of the patient, to titrate supplemental oxygen supplied by a source of supplemental oxygen for delivery to the patient based on the signal, and to continuously monitor the blood oxygen saturation level of the patient based on the signal.

In some embodiments, the instructions stored in the memory device may be executable by the processor may be executable by the processor to cause the processor to receive a signal from a sensor indicative of a measured supplemental oxygen of the patient, to titrate supplemental oxygen supplied by a source of supplemental oxygen for delivery to the patient based on the signal, and to continuously monitor the supplemental oxygen of the patient based on the signal.

In some embodiments, the instructions stored in the memory device may be executable by the processor to cause the processor to titrate supplemental oxygen for delivery to the patient at an initial rate or an initial amount based on the signal. The instructions stored in the memory device may be executable by the processor to cause the processor to compare the measured blood oxygen saturation level of the patient following delivery of supplemental oxygen to the patient at the initial rate or the initial amount to a reference pulse oximetric percentage, and to issue a warning in response to a determination that the measured blood oxygen saturation level of the patient does not meet the reference pulse oximetric percentage. The instructions stored in the memory device may be executable by the processor to cause the processor to titrate supplemental oxygen for delivery to the patient at an increased rate or an increased amount in response to the determination that the measured blood oxygen saturation level of the patient does not meet the reference pulse oximetric percentage. The instructions stored in the memory device may be executable by the processor to cause the processor to compare the measured blood oxygen saturation level of the patient following delivery of supplemental oxygen to the patient at the increased rate or the increased amount to the reference pulse oximetric percentage, and to issue an alarm in response to a determination that the measured blood oxygen saturation level of the patient does not meet the reference pulse oximetric percentage.

In some embodiments, the instructions stored in the memory device may be executable by the processor to cause the processor to titrate supplemental oxygen for delivery to the patient at an initial rate or an initial amount based on the signal. The instructions stored in the memory device may be executable by the processor to cause the processor to compare the measured supplemental oxygen of the patient following delivery of supplemental oxygen to the patient at the initial rate or the initial amount to a reference pulse oximetric percentage, and to issue a warning in response to a determination that the measured supplemental oxygen of the patient does not meet the reference pulse oximetric percentage. The instructions stored in the memory device may be executable by the processor to cause the processor to titrate supplemental oxygen for delivery to the patient at an increased rate or an increased amount in response to the determination that the measured supplemental oxygen of the patient does not meet the reference pulse oximetric percentage. The instructions stored in the memory device may be executable by the processor to cause the processor to compare the measured supplemental oxygen of the patient following delivery of supplemental oxygen to the patient at the increased rate or the increased amount to the reference pulse oximetric percentage, and to issue an alarm in response to a determination that the measured supplemental oxygen of the patient does not meet the reference pulse oximetric percentage.

According to yet another aspect of the present disclosure, a method of delivering supplemental oxygen to a patient may include receiving, by a controller of an automated oxygen monitoring system, a signal provided by a sensor coupled to the oxygen monitoring system that is indicative of a measured blood oxygen saturation level of the patient, titrating, by the controller, supplemental oxygen supplied by a source of supplemental oxygen for delivery to the patient based on the signal, and continuously monitoring, by the controller, the measured blood oxygen saturation level of the patient based on the signal.

In some embodiments, the signal may be indicative of a measured supplemental oxygen of the patient. Additionally, in some embodiments, continuously monitoring, by the controller, may include continuously monitoring supplemental oxygen of the patient based on the signal.

In some embodiments, the method may include titrating, by the controller, supplemental oxygen for delivery to the patient at an initial rate or an initial amount based on the signal. The method may include comparing, by the controller, the measured blood oxygen saturation level of the patient following delivery of supplemental oxygen to the patient at the initial rate or the initial amount to a reference pulse oximetric percentage, and issuing, by the controller, a warning in response to a determination that the measured blood oxygen saturation level of the patient does not meet the reference pulse oximetric percentage. The method may include titrating, by the controller, supplemental oxygen for delivery to the patient at an increased rate or an increased amount in response to the determination that the measured blood oxygen saturation level of the patient does not meet the reference pulse oximetric percentage. The method may include comparing, by the controller, the measured blood oxygen saturation level of the patient following delivery of supplemental oxygen to the patient at the increased rate or the increased amount to the reference pulse oximetric percentage, and issuing, by the controller, an alarm in response to a determination that the measured blood oxygen saturation level of the patient does not meet the reference pulse oximetric percentage.

In some embodiments, the method may include titrating, by the controller, supplemental oxygen for delivery to the patient at an initial rate or an initial amount based on the signal. The method may include comparing, by the controller, the measured supplemental oxygen of the patient following delivery of supplemental oxygen to the patient at the initial rate or the initial amount to a reference pulse oximetric percentage, and issuing, by the controller, a warning in response to a determination that the measured supplemental oxygen of the patient does not meet the reference pulse oximetric percentage. The method may include titrating, by the controller, supplemental oxygen for delivery to the patient at an increased rate or an increased amount in response to the determination that the measured blood oxygen saturation level of the patient does not meet the reference pulse oximetric percentage. The method may include comparing, by the controller, the measured supplemental oxygen of the patient following delivery of supplemental oxygen to the patient at the increased rate or the increased amount to the reference pulse oximetric percentage, and issuing, by the controller, an alarm in response to a determination that the measured blood oxygen saturation level of the patient does not meet the reference pulse oximetric percentage or a determination that the measured supplemental oxygen has exceeded titration parameters.

These and other features of the present disclosure will become more apparent from the following description of the illustrative embodiments.

### BRIEF DESCRIPTION OF THE DRAWINGS

The invention described herein is illustrated by way of example and not by way of limitation in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements.

FIG. 1 is a partial diagrammatic view of an arrangement for delivering supplemental oxygen to a patient;

FIG. 2 is a partial diagrammatic view of a portion of the arrangement depicted in FIG. 1;

FIG. 3 is a partial diagrammatic view of another portion of the arrangement depicted in FIG. 1;

FIG. 4 is a diagrammatic view of a control system included in the arrangement shown in FIG. 1;

FIG. 5 is a simplified flowchart of a method that may be performed by the control system depicted in FIG. 4;

FIG. 6 is a simplified flowchart of one portion of a method that may be performed by the control system during performance of the method illustrated in FIG. 5; and

FIG. 7 is a simplified flowchart of another portion of the method of FIG. 6.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features, such as those representing devices, modules, instructions blocks and data elements, may be shown in specific arrangements and/or orderings for ease of description. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

In some embodiments, schematic elements used to represent blocks of a method may be manually performed by a user. In other embodiments, implementation of those schematic elements may be automated using any suitable form of machine-readable instruction, such as software or firmware applications, programs, functions, modules, routines, processes, procedures, plug-ins, applets, widgets, code fragments and/or others, for example, and each such instruction may be implemented using any suitable programming language, library, application programming interface (API), and/or other software development tools. For instance, in some embodiments, the schematic elements may be implemented using Java, C++, and/or other programming languages. Similarly, schematic elements used to represent data or information may be implemented using any suitable electronic arrangement or structure, such as a register, data store, table, record, array, index, hash, map, tree, list, graph, file (of any file type), folder, directory, database, and/or others, for example.

Further, in the drawings, where connecting elements, such as solid or dashed lines or arrows, are used to illustrate a connection, relationship, or association between or among two or more other schematic elements, the absence of any such connection elements is not meant to imply that no connection, relationship, or association can exist. In other words, some connections, relationships, or associations between elements may not be shown in the drawings so as not to obscure the disclosure. In addition, for ease of illustration, a single connecting element may be used to represent multiple connections, relationships, or associations between elements. For example, where a connecting element represents a communication of signals, data or instructions, it should be understood by those skilled in the art that such element may represent one or multiple signal paths (e.g., a bus), as may be needed, to effect the communication.

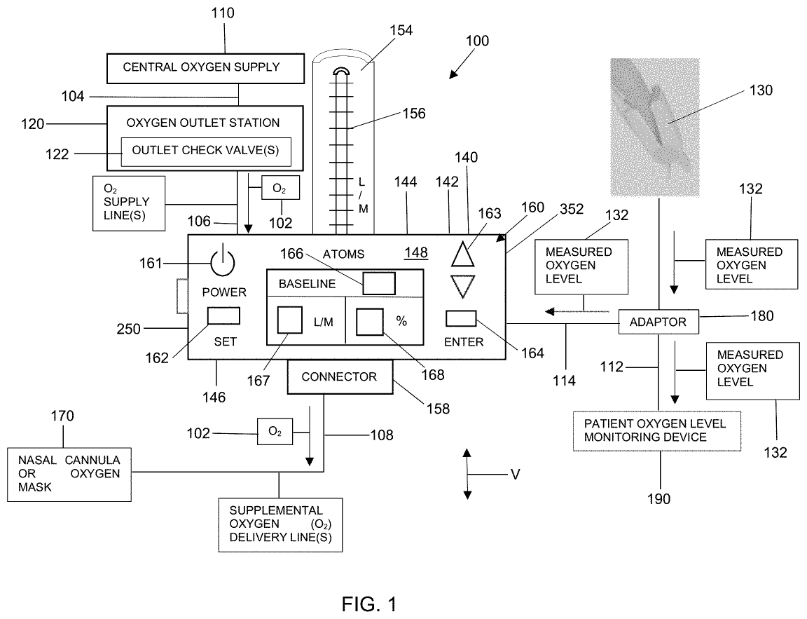

Referring now to FIG. 1, an illustrative arrangement 100 to deliver supplemental oxygen 102 to a patient includes a source of supplemental oxygen 110, an oxygen outlet station 120 having at least one valve 122, a sensor 130, and an automated oxygen monitoring system 140. The at least one valve 122 is fluidly coupled to the supplemental oxygen source 110 and configured to permit flow of supplemental oxygen 102 therethrough. For the purposes of the present disclosure, supplemental oxygen 102 may be interchangeably referred to, associated with, or otherwise correspond to, a blood oxygen saturation level of the patient. Additionally, for the purposes of the present disclosure, a measured blood oxygen level of the patient may be defined as a percentage of hemoglobin molecules bound by oxygen. In any case, the sensor 130 is directly attachable to the patient and configured to provide a signal indicative of a measured supplemental oxygen 132 of the patient. The automated oxygen monitoring system or oxygen saturation monitor 140 is communicatively coupled to the sensor 130 and in fluid communication with a tube 170 adapted for non-invasive insertion into the patient. Hereinafter, for the purposes of simplicity, the automated oxygen monitoring system 140 may be referred to as the ATOMS 140. That reference is notably contained in several of FIGS. 1-7. For the purposes of the present disclosure, the acronym ATOMS stands for automated titratable oxygen monitoring system.

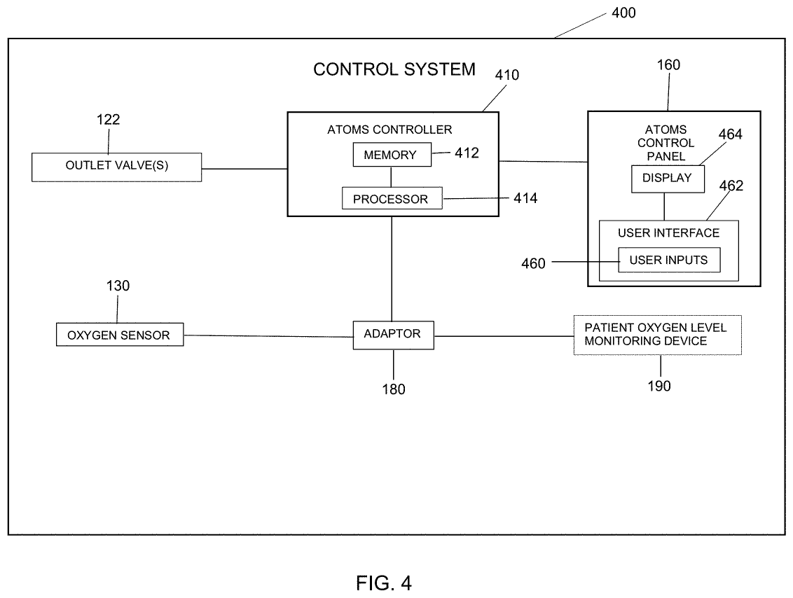

In the illustrative embodiment, the ATOMS 140 is in fluid communication with the source 110 of supplemental oxygen 102 to receive the flow of supplemental oxygen 102 through the at least one valve 122. The ATOMS 140 includes a controller 410 (see FIG. 4) having a memory device 412 and a processor 414 coupled thereto. As described in greater detail below, the memory device 412 has instructions stored therein that are executable by the processor 414 to cause the processor 414 to receive the signal from the sensor 130, to titrate supplemental oxygen 102 for delivery to the patient through the tube 170 based on the signal, and to continuously monitor the supplemental oxygen 132 of the patient based on the signal. As will be appreciated from the discussion that follows, the ATOMS 140 provides proactive and continuous monitoring of a patient's supplemental oxygen during delivery of supplemental oxygen 102 to the patient, thereby avoiding periodic measurements performed at the discretion of hospital personnel and/or caregiver(s). In addition, the ATOMS 140 enables automatic titration of supplemental oxygen for delivery to the patient while avoiding manual adjustments that may be required under existing protocols.

In many conventional configurations, hospital personnel and/or caregiver(s) measure a patient's supplemental oxygen level and selectively deliver supplemental oxygen to the patient according to an at least partially discretionary procedure. That procedure may entail, as a first step, an initial measurement of the patient's blood oxygen level using a pulse oximeter, for instance. Following that measurement, the caregiver (e.g., a nurse) may make a determination that the measured supplemental oxygen of the patient falls below an acceptance threshold warranting delivery of supplemental (i.e., supra-atmospheric) oxygen. Thereafter, the caregiver may deliver supplemental oxygen to the patient at an initial flow rate expected to increase the supplemental oxygen of the patient and, subsequently, the oxygen content. The initial flow rate selected by the caregiver, as well as the time period during which supplemental oxygen is delivered to the patient, may be subject to the discretion of the caregiver.

At some point subsequent to the initial delivery of supplemental oxygen, the caregiver measures the patient's supplemental oxygen to determine if the level has increased. In the event the patient's measured supplemental oxygen has increased to the threshold mentioned above, the caregiver may continue to deliver supplemental oxygen to the patient at the initial flow rate or cease further delivery of supplemental oxygen. However, if the desired increase has not occurred, the caregiver may increase, dramatically in some cases, the flow rate of supplemental oxygen to the patient. The increased flow rate of supplemental oxygen and the duration over which supplemental oxygen is delivered at the increased flow rate are once again subject to the caregiver's discretion.

Following delivery of supplemental oxygen to the patient at the increased flow rate, the caregiver may again measure the patient's blood oxygen level to determine if the level has increased to the threshold. If that increase has been achieved, the caregiver may continue to deliver supplemental oxygen to the patient at the increased flow rate or cease further delivery of supplemental oxygen. If that increase has not occurred, the caregiver may decide to alert other hospital personnel or caregivers (e.g., one or more physicians).

It should be appreciated that the procedure described above involves periodic, non-continuous measurement of the patient's supplemental oxygen. Indeed, depending on the setting in which care is administered (e.g., an intensive care unit, an emergency room, a post-surgery location, etc.) to the patient, the measurement frequency of the patient's supplemental oxygen may vary. Furthermore, it should be evident that the aforementioned procedure entails, and is characterized by, manual selection and adjustment of the flow rate or amount of supplemental oxygen to be delivered to the patient. Such manual processes simply fail to provide an automated means for continuously monitoring a patient's blood oxygen level and titrating supplemental oxygen for delivery to a patient.

In the illustrative embodiment, the arrangement 100 includes an adaptor 180 communicatively coupled to the sensor 130 and the ATOMS 140. As discussed below, the adaptor 180 is configured to transmit the signal provided by the sensor 130 to the processor 414 of the ATOMS 140. Furthermore, the illustrative arrangement 100 includes an oxygen saturation monitor 190 communicatively coupled to the adaptor 180. The adaptor 180 is configured to transmit the signal provided by the sensor 130 to the oxygen saturation monitor 190 to permit continuous measurement of the supplemental oxygen of the patient via the oxygen saturation monitor 190, as discussed below. The illustrative oxygen saturation monitor 190 is provided separately from the ATOMS 140.

In some situations, as both the ATOMS 140 and the oxygen saturation monitor 190 receive the signal provided by the sensor 130, those devices may provide redundant means for continuous measurement and monitoring of the patient's supplemental oxygen. Furthermore, in some situations, the oxygen saturation monitor 190 may be a supplemental oxygen monitor. However, in other situations, it should be appreciated that the oxygen saturation monitor 190 may be omitted from the patient care setting and the arrangement 100.

The illustrative source of supplemental oxygen 110 includes, or is otherwise embodied as, any source or collection of sources for supplying supplemental oxygen 102 to the patient. In some embodiments, the source 110 may form a portion of, or otherwise be coupled to, a central oxygen supply located in a hospital or other patient care setting. Of course, it should be appreciated that the source 110 may include one or more tanks, supply banks, manifolds, distribution chambers, or the like for the storage of supplemental oxygen 102. In any case, the supplemental oxygen source 110 is fluidly coupled to the outlet station 120 by a fluidic coupling 104 to permit fluid communication of supplemental oxygen 102 from the source 110 to the at least one valve 122 of the outlet station 120. The fluidic coupling 104 may include one or more pipes, conduits, elbow sections, fluid lines, or the like.

The illustrative outlet station 120 includes, or is otherwise embodied as, one or more supply ports, terminals, or outlets through which supplemental oxygen 102 may be delivered to a patient in a location remote from the supplemental oxygen source 110. As indicated above, the outlet station 120 includes the at least one valve 122 fluidly coupled to the source 110. In some embodiments, the at least one valve 122 includes one or more check valves (e.g., primary and secondary check valves) configured to permit one-way flow of supplemental oxygen 102 from the source 110 to the ATOMS 140. Additionally, in some embodiments, the at least one valve 122 includes one or more solenoid valves controllable in response to control signals provided by the controller 410, as discussed below with reference to FIG. 4. Regardless, the outlet station 120 and the at least one valve 122 are fluidly coupled to the ATOMS 140 by a fluidic coupling 106. The fluidic coupling 106 may include one or more pipes, conduits, elbow sections, fluid lines, or the like.

In the illustrative embodiment, the outlet station 120 includes one or more safety systems (not shown) that employ standardized quick connectors and/or adaptors. Such systems may include and/or employ any one or more of the following: a diameter index safety system (DISS), a Puritan-Bennett quick-connect system, an Ohio Diamond system, a NCG/Chemetron system, a Hansen Schrader system, and a Canadian Liquid Air system. Of course, it should be appreciated that in other embodiments, other suitable quick connectors and/or adaptors may be utilized.

The illustrative sensor 130 includes, or is otherwise embodied as, any device or collection of devices capable of providing a signal indicative of a measured supplemental oxygen 132 of the patient. In some embodiments, the illustrative sensor 130 is provided in the form of a pulse oximeter directly attachable to the patient (e.g., a fingertip, a nose, etc. of the patient) and capable of measuring oxygen saturation of the patient's blood. In one example, the pulse oximeter is capable of measuring oxygen saturation of the patient's blood by a transmissive pulse oximetry approach. In another example, the pulse oximeter is capable of measuring oxygen saturation of the patient's blood by a reflectance pulse oximetry approach.

The illustrative tube 170 includes, or is otherwise embodied as, any device or collection of devices configured for non-invasive insertion into the patient (e.g., a nose or mouth of the patient) to deliver supplemental oxygen titrated by the ATOMS 140 to the patient. In some embodiments, the tube 170 may be provided in the form of a cannula, such as an intravenous cannula or a nasal cannula, for example. Additionally, in some embodiments, the tube 170 may be provided in the form of a pipe or pipette, a straw, a duct, or the like. The tube 170 is illustratively fluidly coupled to the ATOMS 140 by a fluidic coupling 108 to permit flow of supplemental oxygen 102 from the ATOMS 140 to the tube 170 for delivery to the patient. The fluidic coupling 108 may include one or more pipes, conduits, elbow sections, fluid lines, or the like.

The illustrative adaptor 180 includes, or is otherwise embodied as, any device or collection of devices configured to transmit the signal provided by the sensor 130 to the processor 414 of the ATOMS 140 and to the oxygen saturation monitor 190 as indicated above. In some embodiments, the adaptor 180 is provided in the form of a tee connector. In other embodiments, the adaptor 180 may be provided in another suitable form, such as a universal connector, a board-to-board connector, a cable/wire-to-cable/wire connector, a cable/wire-to-board connector, a Wago connector, a T-fitting, or the like. The adaptor 180 is illustratively communicatively coupled to the sensor 130 by at least one cable 110 to receive the signal from the sensor 130. The adaptor 180 is also communicatively coupled to the oxygen saturation monitor 190 by at least one cable 112 to transmit the signal from the sensor 130 to the device 190. Additionally, the adaptor 180 is communicatively coupled to the ATOMS 140 by at least one cable 114 to transmit the signal from the sensor 130 to the ATOMS 140.

The illustrative oxygen saturation monitor 190 includes, or is otherwise embodied as, any device or collection of devices capable of monitoring the supplemental oxygen of the patient based on the signal provided by the sensor 130. In some embodiments, the oxygen saturation monitor 190 is configured to provide a visual indication of the patient's blood oxygen saturation level corresponding to a waveform graphically represented on a plot. In one example, a plot representing the waveform may include one axis associated with a partial pressure percentage of arterial oxygen and another axis associated with a percentage of pulse oximetry saturation. In any case, it should be appreciated that the oxygen saturation monitor 190 may include a display to visualize the indication and issue any number of visual and/or audible alarms, alerts, or warnings to prompt action by hospital personnel or caretakers.

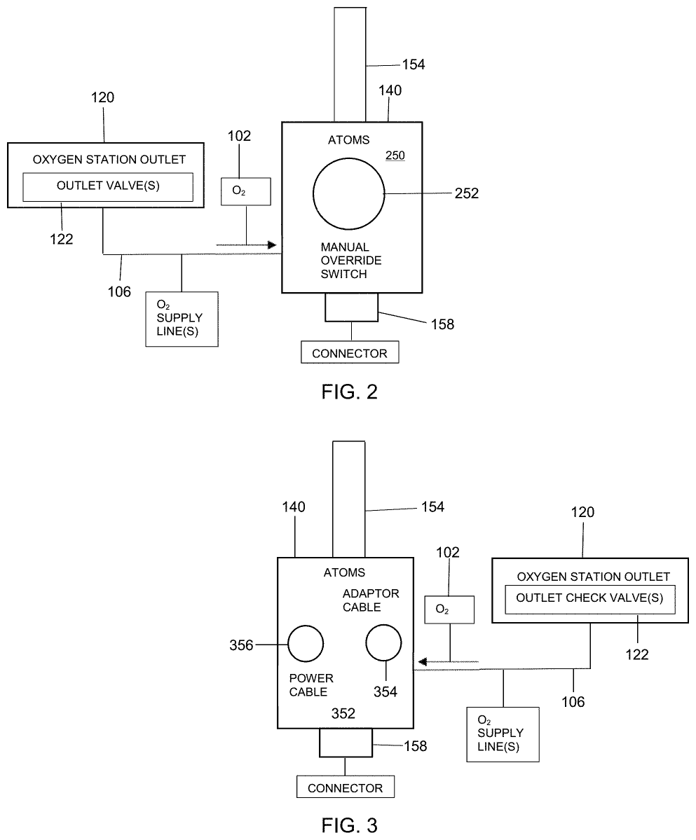

In the illustrative embodiment, the ATOMS 140 includes a housing 142, a gage 154, a connector 158, and a control panel 160. The gage 154 is coupled to the housing 142 such that the gage 154 extends upwardly from a top face 144 of the housing 142 in a vertical direction indicated by arrow V. The connector 158 is coupled to the housing 142 such that the connector 158 extends downwardly from a bottom face 146 of the housing 142 in the vertical direction V. The control panel 160 is coupled to the housing 142 and disposed on a front face 148 thereof. Side faces 250 (see FIG. 2) and 352 (see FIG. 3) of the housing 142 are arranged opposite one another. In some embodiments, a rear face (not shown) of the housing 142 is arranged in direct contact with a supporting structure (e.g., a wall) so that the ATOMS 140 is supported by the supporting structure.

The illustrative gage 154 includes, or is otherwise embodied as, an observable indicator of the amount of supplemental oxygen 102 delivered to the patient using the ATOMS 140. The gage 154 includes a plurality of markers 156 indicative of the level of supplemental oxygen 102 provided by the ATOMS 140. More specifically, in the illustrative embodiment, each of the markers 156 is indicative of a volumetric flow rate (e.g., in liter/min) of supplemental oxygen 102 delivered to the patient using the ATOMS 140. In some embodiments, the gage 154 may be omitted.

The illustrative connector 158 includes, or is otherwise embodied as, any feature or collection of features configured for interaction with the tube 170 (i.e., through the fluidic coupling 108) to permit delivery of supplemental oxygen 102 from the ATOMS 140 to the patient via the tube 170. In some embodiments, the connector 158 includes a post, stem, spout, outlet port, or the like that is sized for receipt by the fluidic coupling 108. Of course, in other embodiments, it should be appreciated that the connector 158 may include another suitable feature to facilitate interaction with the fluidic coupling 108.

In the illustrative embodiment, the control panel 160 includes a plurality of user inputs 460 (which are diagrammatically depicted in FIG. 4). The inputs 460 are provided as part of, or otherwise incorporated into, a user interface 462. Each of the user inputs 460 is manually manipulatable to provide user input to the controller 410. The user inputs 460 illustratively include a power button 161, a set button 162, adjustment arrows 163, and an enter button 164. It should be appreciated, however, that in some embodiments, the user inputs 460 may include features in addition to, and/or as an alternative to, those discussed above.

The illustrative power button 161 is manually manipulatable by a user to power on/off the ATOMS 140. The illustrative set button 162 is manually manipulatable by a user to select a desired setpoint (e.g., a volumetric flow rate in liter/min) for the delivery of titrated supplemental oxygen 102 to the patient via the ATOMS 140. The adjustment arrows 163 are illustratively manipulatable by a user to increase or decrease the rate (e.g., in liter/min) at which titrated supplemental oxygen 102 is delivered to the patient by the ATOMS 140, which may alter the indicators 166, 167 described below. In some embodiments, rate adjustments may be performed with the arrows 163 prior to selection of a particular setpoint using the set button 162. The enter button 164 is manually manipulatable by a user to confirm, validate, and/or establish a command or input provided to the ATOMS 140 by a user, at least in some embodiments. Additionally, in some embodiments, the enter button 164 may be used in conjunction with the set button 162 to select and confirm a desired setpoint for the delivery of titrated supplemental oxygen 102 by the ATOMS 140.

In the illustrative embodiment, the control panel 160 includes a plurality of indicators in addition to the user inputs 460. More specifically, the control panel 160 includes a baseline indicator 166, an oxygen flow indicator 167, and a supplemental oxygen indicator 168. The baseline indicator 166 is illustratively configured to indicate the amount (e.g., the volumetric flow rate) of supplemental oxygen 102 supplied to the ATOMS 140 from the outlet station 120. In some embodiments, adjustments to the amount indicated by the baseline indicator may be preceded by selection of a setpoint using the button 162. Additionally, in some embodiments, the baseline indicator 166 may include, or otherwise be embodied as, a default indication based on a room air setting in the particular patient care environment. The oxygen flow indicator 167 is illustratively configured to indicate the amount (e.g., the volumetric flow rate in liter/min) of supplemental oxygen 102 titrated by the ATOMS 140 for delivery to the patient. The supplemental oxygen indicator 168 is illustratively configured to indicate the measured blood oxygen saturation level (e.g., as a pulse oximetric saturation percentage) of the patient. The supplemental oxygen indicated by the indicator 168 is illustratively based on the signal produced by the sensor 130 and transmitted to the ATOMS 140 through the adaptor 180.

It should be appreciated that in the illustrative embodiment, no component of the ATOMS 140 is arranged in direct contact with the patient. Rather, the ATOMS 140 is configured for indirect interaction with the patient through the sensor 130, the tube 170, and/or the adaptor 180. In some configurations, aside from the ATOMS 140, a number of devices included in the illustrative arrangement 100 may be provided as existing and/or standard components located in a hospital facility or caretaker setting. In one example, the source of supplemental oxygen 110, the outlet station 120 and the at least one valve 122, the sensor 130, and the oxygen level monitoring device 190 may be provided as existing components located in a hospital facility or caretaker setting. In such configurations, the ATOMS 140 may be provided as a standalone unit configured for connection to those existing components to provide the arrangement 100. Stated differently, the ATOMS 140 may be retrofitted into an environment in a hospital facility or healthcare setting such that the ATOMS 140 is integrated with existing components of that environment, thereby facilitating reduced design complexity and cost and increased ease of manufacturing, at least in some embodiments.

Referring now to FIG. 2, the ATOMS 140 is illustratively depicted facing the side face 250 thereof. In the illustrative embodiment, an override switch 252 is disposed on the side face 250 of the ATOMS. The override switch 252 is manually manipulatable by a user to selectively cease, increase, or decrease delivery of supplemental oxygen 102 to the patient through the ATOMS 140. In some embodiments, manipulation of the switch 252 may cause, or otherwise be associated with, closure of the at least one valve 122 to prevent flow of supplemental oxygen 102 to the ATOMS 140. In other embodiments, manipulation of the switch 252 may cause, or otherwise be associated with, a flow blockage or obstruction internally within the ATOMS 140 to prevent supplemental oxygen 102 from being delivered to the patient through the ATOMS 140.

Referring now to FIG. 3, the ATOMS 140 is illustratively depicted facing the side face 352 thereof. In the illustrative embodiment, the side face 352 is formed to include a port 354 and a port 356. The port 354 is sized to accommodate the cable 114 that communicatively couples the adaptor 180 to the ATOMS 140. The port 356 is sized to accommodate a power cable (not shown) of the ATOMS 140. It should be appreciated that in some embodiments, the side face 352 may be formed to include features in addition to, or as an alternative to, those discussed above.

Referring now to FIG. 4, in the illustrative embodiment, the arrangement 100 includes a control system 400. The control system 400 includes, or is otherwise embodied as, a collection of components cooperatively configured to control operation and thereby provide various functionalities of the arrangement 100. The illustrative control system 400 includes the controller 410 and a number of devices each communicatively coupled to the controller 410, which include, but are not limited to, the following: the at least one outlet valve 122, the adaptor 180, and the control panel 160 of the ATOMS 140. Furthermore, as described above, each of the sensor 130 and the oxygen level monitoring device 190 are communicatively coupled to the controller 410 through the adaptor 180. Although not expressly depicted in FIG. 4, it should be appreciated that the control system 400 may include components in addition to, or as an alternative to, those mentioned above.

The memory device 412 of the illustrative controller 410 may be embodied as any type of volatile (e.g., dynamic random access memory (DRAM), etc.) or non-volatile memory capable of storing data therein. Volatile memory may be embodied as a storage medium that requires power to maintain the state of data stored by the medium. Non-limiting examples of volatile memory may include various types of random access memory (RAM), such as dynamic random access memory (DRAM) or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4 (these standards are available at www.jedec.org). Such standards (and similar standards) may be referred to as DDR-based standards and communication interfaces of the storage devices that implement such standards may be referred to as DDR-based interfaces.

In some embodiments, the memory device 412 may be embodied as a block addressable memory, such as those based on NAND or NOR technologies. The memory device 412 may also include future generation nonvolatile devices, such as a three dimensional crosspoint memory device (e.g., Intel 3D XPoint™ memory), or other byte addressable write-in-place nonvolatile memory devices. In some embodiments, the memory device 412 may be embodied as, or may otherwise include, chalcogenide glass, multi-threshold level NAND flash memory, NOR flash memory, single or multi-level Phase Change Memory (PCM), a resistive memory, nanowire memory, ferroelectric transistor random access memory (FeTRAM), anti-ferroelectric memory, magnetoresistive random access memory (MRAM) memory that incorporates memristor technology, resistive memory including the metal oxide base, the oxygen vacancy base and the conductive bridge Random Access Memory (CB-RAM), or spin transfer torque (STT)-MRAM, a spintronic magnetic junction memory based device, a magnetic tunneling junction (MTJ) based device, a DW (Domain Wall) and SOT (Spin Orbit Transfer) based device, a thyristor based memory device, or a combination of any of the above, or other memory. The memory device may refer to the die itself and/or to a packaged memory product. In some embodiments, 3D crosspoint memory (e.g., Intel 3D XPoint™ memory) may comprise a transistor-less stackable cross point architecture in which memory cells sit at the intersection of word lines and bit lines and are individually addressable and in which bit storage is based on a change in bulk resistance.

The processor 414 of the illustrative controller 410 may be embodied as, or otherwise include, any type of processor, controller, or other compute circuit capable of performing various tasks such as compute functions and/or controlling the functions of the ATOMS 140. For example, the processor 414 may be embodied as a single or multi-core processor(s), a microcontroller, or other processor or processing/controlling circuit. In some embodiments, the processor 414 may be embodied as, include, or otherwise be coupled to an FPGA, an application specific integrated circuit (ASIC), reconfigurable hardware or hardware circuitry, or other specialized hardware to facilitate performance of the functions described herein. Additionally, in some embodiments, the processor 414 may be embodied as, or otherwise include, a high-power processor, an accelerator co-processor, or a storage controller. In some embodiments still, the processor 414 may include more than one processor, controller, or compute circuit.

The illustrative control panel 160 includes a display 464 and the user interface 462 communicatively coupled thereto. As indicated above, the user interface 462 includes the user inputs 460. The display 464 illustratively includes, or is otherwise embodied as, any device or collection of devices configured to output or display various indications, messages, and/or prompts to a user, which may be generated by the control system 400 as described in greater detail below. In some embodiments, the display 464 may include, among other things, the various indicators discussed above (i.e., the baseline indicator 166, the SpO₂ flow indicator 167, and the pulse oximetry indicator 168). In any case, as should be evident from the discussion above, the illustrative user interface 462 is configured to provide various inputs to the control system 400, which may include actions performed (i.e., manual manipulation) by a user using the inputs 460.

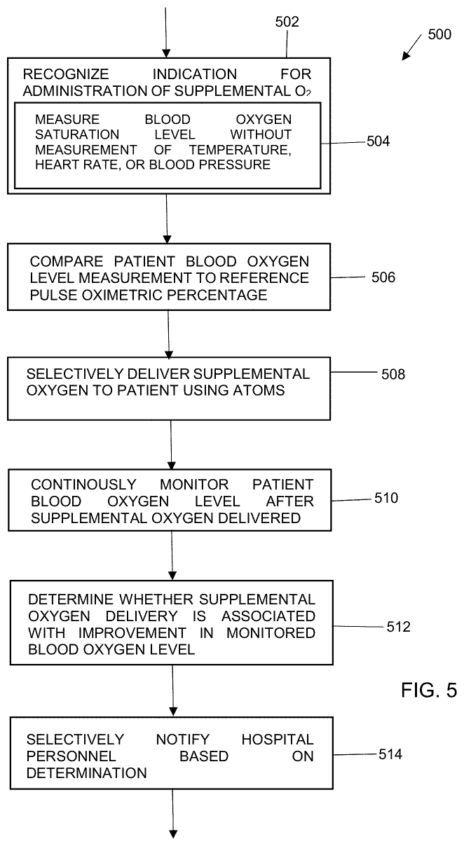

Referring now to FIG. 5, an illustrative method 500 may be performed by the controller 410 of the ATOMS 140. The method 500 includes, or is otherwise embodied as, a set of instructions stored in the memory device 412 that are executable by the processor 414 to control operation of the ATOMS 140 and the arrangement 100, at least in some embodiments. The method 500 corresponds to, or is otherwise associated with, performance of the blocks described below in the illustrative sequence of FIG. 5. It should be appreciated, however, that the method 500 may be performed in one or more sequences different from the illustrative sequence. Furthermore, in some embodiments, one or more of the blocks described below may be performed by an operator in conjunction with, or as an alternative to, automated performance of those blocks by the controller 410.

The illustrative method 500 begins with block 502. In block 502, based on receipt of the signal produced by the sensor 130 indicative of the measured blood oxygen saturation level of the patient, the controller 410 recognizes an indication for administration of supplemental oxygen. It should be appreciated that in some cases, an indication recognized in block 502 by the controller 410 may be associated with a patient not already receiving supplemental oxygen, and that such recognition may occur while performing checks at predetermined intervals in an emergency department setting, for example. Additionally, it should be appreciated that in other cases, an indicator recognized in block 502 by the controller 410 may be associated with a patient already receiving supplemental oxygen, and that such recognition may occur while performing checks at a patient's home, an outside hospital setting, a post-surgery setting, or upon hospital admission following evaluation in an emergency department, for example. In any case, as mentioned above, the signal may be in the form of, or otherwise correspond to, a pulse oximetric oxygen saturation percentage. In any case, to perform block 502, at least in some embodiments, the controller 410 illustratively performs block 504. In block 504, the controller 410 measures (i.e., based on the signal from the sensor 130) the blood oxygen saturation level without performing a measurement of other physiological characteristics of the patient, such as temperature, heart rate, and/or blood pressure, just to name a few. Of course, in other embodiments, block 504 may involve measurement of the patient's blood oxygen saturation level without measurement of other physiological conditions. Upon completion of block 504, the method 500 subsequently proceeds to block 506.

In block 506 of the illustrative method 500, the controller 410 compares the measured blood oxygen saturation level of the patient to a reference value (e.g., a reference pulse oximetric oxygen saturation percentage). In some embodiments, the reference value or reference pulse oximetric oxygen saturation percentage may represent a normal or acceptable blood oxygen saturation level for any given patient. In other embodiments, the reference value or reference pulse oximetric oxygen saturation percentage may represent a normal or acceptable blood oxygen saturation level for a particular patient based on certain factors unique to the patient. In one example, the reference pulse oximetric oxygen saturation percentage may be 92% (SpO₂) or greater, such as 95%. In another example, the reference oximetric oxygen saturation percentage may be 88% (SpO₂) or greater, such as 90%, which may be a reference value for a patient suffering from chronic obstructive pulmonary disease (COPD). Regardless, from block 506, the method 500 subsequently proceeds to block 508.

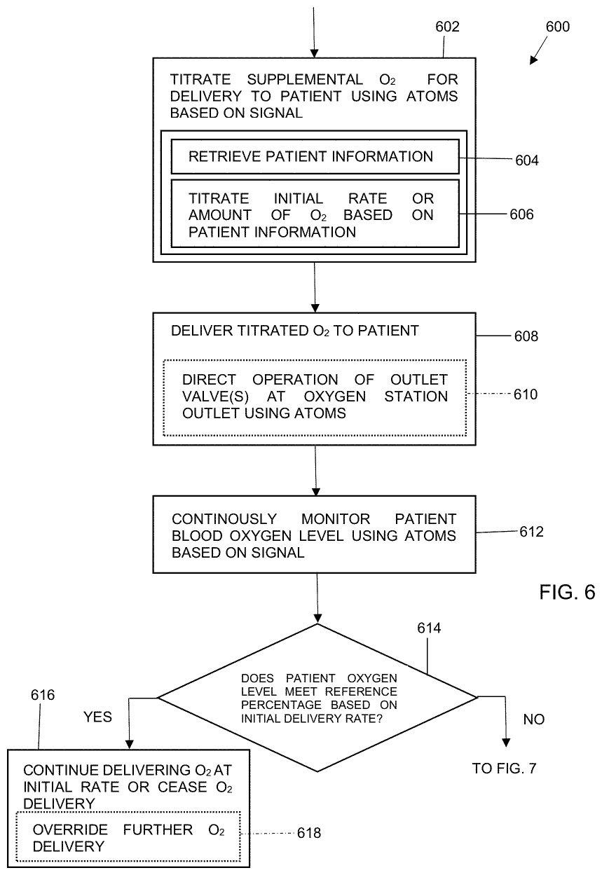

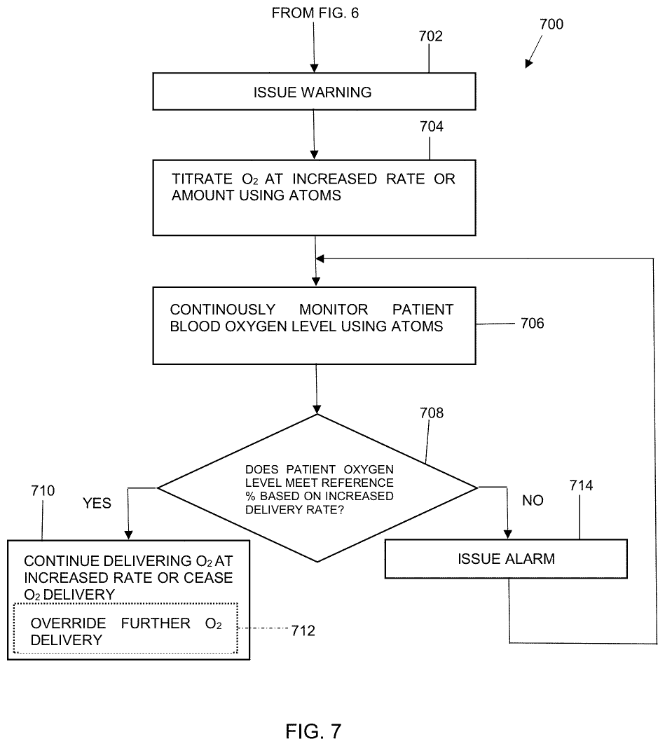

In block 508 of the illustrative method 500, the controller 410 selectively delivers supplemental oxygen 102 to the patient using the ATOMS 140 based on the comparison performed in block 506. To do so, at least in some embodiments, the controller 410 performs the method 600 described below with reference to FIGS. 6 and 7. For the purposes of the present disclosure, delivery of supplemental oxygen 102 to the patient in block 508, and performance of the blocks of the method 600 to complete block 508, presumes the comparison performed in block 506 warrants delivery of supplemental oxygen 102 to the patient. Put another way, delivery of supplemental oxygen 102 in block 508 and the performance of the method 600 presumes that the patient needs supplemental oxygen 102. From block 508, the method 500 subsequently proceeds to block 510.

In block 510 of the illustrative method 500, the controller 410 continuously monitors the blood oxygen level of the patient after initial delivery of supplemental oxygen 102 to the patient. In some embodiments, to perform block 510, the controller 410 may perform one or more of the blocks of the method 600 described below. It should be appreciated that the controller 410 may perform block 510 based on the signal generated by the sensor 130 and any other input provided by the oxygen saturation monitor 190, at least in some embodiments. From block 510, the method 500 subsequently proceeds to block 512.

In block 512 of the illustrative method 500, the controller 410 determines whether the initial delivery of supplemental oxygen 102 to the patient is associated with an improvement in the monitored blood oxygen level of the patient. In some embodiments, to perform block 512, the controller 410 may perform one or more of the blocks of the method 600 described below. From block 512, the method 500 subsequently proceeds to block 514.

In block 514 of the illustrative method 500, the controller 410 selectively notifies hospital personnel (or other caretakers depending on the patient care setting) based on the determination made in block 512. In some embodiments, to perform block 514, the controller 410 may perform one or more of the blocks of the method 600 described below. As will be apparent from the discussion that follows, notification of hospital personnel or other caretakers in block 514 presumes that delivery of supplemental oxygen 102 to the patient does not result in an increase of the patient's monitored blood oxygen level to a normal or acceptable reference value.

Referring now to FIG. 6, the illustrative method 600 may be performed by the controller 410 of the ATOMS 140. The method 600 includes, or is otherwise embodied as, a set of instructions stored in the memory device 412 that are executable by the processor 414 to control operation of the ATOMS 140 and the arrangement 100, at least in some embodiments. The method 600 corresponds to, or is otherwise associated with, performance of the blocks described below in the illustrative sequence of FIG. 6. It should be appreciated, however, that the method 600 may be performed in one or more sequences different from the illustrative sequence. Furthermore, in some embodiments, one or more of the blocks described below may be performed by an operator in conjunction with, or as an alternative to, automated performance of those blocks by the controller 410.

The illustrative method 600 begins with block 602. Presuming a need for supplemental oxygen 102 exists, in block 602, the controller 410 titrates supplemental oxygen for delivery to the patient based on the signal provided by the sensor 130. For the purposes of the present disclosure, titration performed by the ATOMS 140 refers to continuous measurement and adjustment of a dosage (e.g., a flow rate) of, or any constituent contained in a dosage of, supplemental oxygen 102. It should be appreciated that any dosage adjustment of supplemental oxygen 102 by the ATOMS 140 in the course of the aforementioned titration may be automatically performed based on the patient's response to the initial delivery of supplemental oxygen. In any case, in the illustrative embodiment, to perform block 602, the controller 410 performs blocks 604 and 606. In block 604, the controller 410 retrieves information specific to the particular patient. The controller 410 may retrieve the patient specific information from a new baseline input, database, library, electronic medical record, electronic history file, or any other suitable repository in which that information is stored and accessible to the controller 410. In block 606, the controller 410 titrates an initial rate or amount of supplemental oxygen 102 for delivery to the patient based on the patient information retrieved in block 604. In some embodiments, the initial rate or amount of supplemental oxygen 102 titrated in block 606 may be based on an initial delivery rate of supplemental oxygen typically associated with a desired improvement in a patient's blood oxygen saturation level, such as an initial delivery rate of 2 liters/min, for example. Following completion of blocks 604 and 606, the method 600 subsequently proceeds to block 608.

In block 608 of the illustrative method 600, the controller 410 delivers supplemental oxygen 102 titrated in the block 602 to the patient via the fluidic coupling 108 and the tube 170. In some embodiments, to perform block 608, the controller 410 may perform block 610. In block 610, the controller 410 directs operation of the at least one valve 122 included in the outlet station 120. To do so, the controller 410 may issue one or more control signals to the at least one valve 122 to cause movement (e.g., opening or closing) thereof to achieve a particular flow rate of supplemental oxygen 102 to the ATOMS 140. In such embodiments, the valve(s) 122 may include, or otherwise be embodied as, any electronically-controlled valve, such as a solenoid valve, for example. In other embodiments, however, block 610 may be omitted. From block 608, the method 600 subsequently proceeds to block 612.

In block 612 of the illustrative method 600, the controller 410 continuously monitors the blood oxygen level of the patient based on the signal provided by the sensor 130 following titration of supplemental oxygen 102 and delivery of supplemental oxygen to the patient in blocks 602 and 608. In some embodiments, the continuous monitoring performed by the controller 410 in block 612 may be based at least partially on input provided to the ATOMS 140 from the oxygen saturation monitor 190. Following block 612, the method 600 subsequently proceeds to block 614.

In block 614 of the illustrative method 600, the controller 410 determines whether the patient's monitored blood oxygen saturation level meets the reference value or the reference pulse oximetric oxygen saturation percentage following delivery of titrated supplemental oxygen 102 to the patient based on the initial rate/amount mentioned above in block 602. If the controller 410 determines in block 614 that the blood oxygen saturation level of the patient meets the reference value or the reference pulse oximetric oxygen saturation percentage, the method 600 subsequently proceeds to block 616. However, if the controller 410 determines in block 614 that the blood oxygen saturation level of the patient does not meet the reference value or the reference pulse oximetric oxygen saturation percentage, the method 600 subsequently proceeds to block 702.

In block 616 of the illustrative method 600, the controller 410 continues delivering supplemental oxygen 102 to the patient based on the initially titrated rate/amount (i.e., from block 602) or ceases further delivery of supplemental oxygen to the patient. In some embodiments, continued delivery of supplemental oxygen 102 or cessation of further delivery in block 616 may be performed based on input provided to the ATOMS 140 by an operator. In one example, in response to the determination in block 614 that the blood oxygen saturation level of the patient meets the reference value or the reference pulse oximetric oxygen saturation percentage, the controller 410 may automatically generate a prompt observable via the user interface 462 and/or the display 464 that requests the operator to (i) provide a command to continue further delivery of supplemental oxygen 102 or (ii) provide a command to cease further supplemental oxygen delivery. Additionally, in some embodiments, to perform block 616, the controller 410 may perform block 618. In block 618, the controller 410 overrides further supplemental oxygen delivery to the patient. In some cases, the controller 410 may perform block 618 in response to manipulation of the override switch 252 by an operator. In other cases, the controller 410 may perform block 618 regardless of any manipulation of the override switch 252 by the operator.

Referring now to FIG. 7, in block 702 of the illustrative method 600, the controller 410 issues a warning observable via the user interface 462 and/or the display 464. The warning may also be accompanied by an auditory indication to an operator. The warning may indicate that the monitored blood oxygen saturation level of the patient does not meet the reference value or the reference pulse oximetric oxygen saturation percentage following delivery of titrated supplemental oxygen 102 to the patient based on the initial rate/amount mentioned above with respect to block 602. From block 702, the method 600 subsequently proceeds to block 704.

In block 704 of the illustrative method 600, the controller 410 titrates an increased amount or increased rate (i.e., relative to the initial rate/amount discussed above with reference to block 602) of supplemental oxygen 102 for delivery to the patient. It should be appreciated that block 704 may include delivery of the increased amount/rate of supplemental oxygen to the patient, at least in some embodiments. Additionally, in some embodiments, the increased amount/rate of titrated supplemental oxygen 102 may be 4 liters/min. In any case, from block 704, the method 600 subsequently proceeds to block 706.

In block 706 of the illustrative method 600, the controller 410 continuously monitors the blood oxygen saturation level of the patient based on the signal provided by the sensor 130 following titration of supplemental oxygen 102 and delivery of supplemental oxygen to the patient in block 704. In some embodiments, the continuous monitoring performed by the controller 410 in block 704 may be based at least partially on input provided to the ATOMS 140 from the oxygen saturation monitor 190. Following block 706, the method 600 subsequently proceeds to block 708.

In block 708 of the illustrative method 600, the controller 410 determines whether the patient's monitored blood oxygen saturation level meets the reference value or the reference pulse oximetric oxygen saturation percentage following delivery of titrated supplemental oxygen 102 to the patient based on the increased rate/amount mentioned above in block 704. If the controller 410 determines in block 708 that the blood oxygen saturation level of the patient meets the reference value or the reference pulse oximetric oxygen saturation percentage, the method 600 subsequently proceeds to block 710. However, if the controller 410 determines in block 708 that the blood oxygen saturation level of the patient does not meet the reference value or the reference pulse oximetric oxygen saturation percentage, the method 600 subsequently proceeds to block 714.

In block 710 of the illustrative method 600, the controller 410 continues delivering supplemental oxygen 102 to the patient based on the increased titrated rate/amount (i.e., from block 704) or ceases further delivery of supplemental oxygen to the patient. In some embodiments, continued delivery of supplemental oxygen 102 or cessation of further delivery in block 710 may be performed based on input provided to the ATOMS 140 by an operator. In one example, in response to the determination in block 708 that the blood oxygen saturation level of the patient meets the reference value or the reference pulse oximetric oxygen saturation percentage, the controller 410 may automatically generate a prompt observable via the user interface 462 and/or the display 464 that requests the operator to (i) provide a command to continue further delivery of supplemental oxygen 102 or (ii) provide a command to cease further supplemental oxygen delivery. Additionally, in some embodiments, to perform block 710, the controller 410 may perform block 712. In block 712, the controller 410 overrides further supplemental oxygen delivery to the patient. In some cases, the controller 410 may perform block 712 in response to manipulation of the override switch 252 by an operator. In other cases, the controller 410 may perform block 712 regardless of any manipulation of the override switch 252 by the operator.

In block 714 of the illustrative method 600, the controller 410 issues an alarm observable via the user interface 462 and/or the display 464. The alarm may also be accompanied by an auditory indication to an operator. The alarm may indicate that the monitored blood oxygen saturation level of the patient does not meet the reference value or the reference pulse oximetric oxygen saturation percentage following delivery of titrated supplemental oxygen 102 to the patient based on the increased rate/amount mentioned above with respect to block 704. Issuance of the alarm in block 714 may include, or otherwise be accompanied by, notification of hospital personnel and/or caretakers. From block 714, the method 600 subsequently returns to block 706.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. An arrangement to deliver supplemental oxygen to a patient, the arrangement comprising:
a source of supplemental oxygen;
an outlet station including at least one valve fluidly coupled to the supplemental oxygen source and configured to permit flow of supplemental oxygen therethrough;
a sensor directly attachable to the patient and configured to provide a signal indicative of a measured blood oxygen saturation level of the patient; and
an automated oxygen monitoring system communicatively coupled to the sensor and in fluid communication with a tube adapted for administration of supplemental oxygen to the patient, wherein the oxygen monitoring system is in fluid communication with the source of supplemental oxygen to receive the flow of supplemental oxygen through the at least one valve, wherein the oxygen monitoring system includes a controller having a memory device and a processor coupled thereto, and wherein the memory device has instructions stored therein that are executable by the processor to cause the processor to receive the signal from the sensor, to titrate supplemental oxygen for delivery to the patient through the tube based on the signal, and to continuously monitor the blood oxygen saturation level of the patient based on the signal.

2. The arrangement of claim 1, further comprising an adaptor communicatively coupled to the sensor and the automated oxygen monitoring system, wherein the adaptor is configured to transmit the signal provided by the sensor to the processor.

3. The arrangement of claim 2, further comprising an oxygen saturation monitor communicatively coupled to the adaptor, wherein the adaptor is configured to transmit the signal provided by the sensor to the oxygen saturation monitor to permit continuous measurement of the blood oxygen saturation level of the patient via the oxygen saturation monitor.

4. The arrangement of claim 3, wherein the oxygen saturation monitor is provided separately from the automated oxygen monitoring system.

5. The arrangement of claim 1, wherein the automated oxygen monitoring system includes an override switch, and wherein the override switch is manually manipulatable by a user to selectively cease delivery of supplemental oxygen to the patient through the oxygen monitoring system.

6. The arrangement of claim 1, wherein the instructions stored in the memory device are executable by the processor to cause the processor to retrieve patient information specific to the patient and to titrate supplemental oxygen for delivery to the patient at an initial rate or an initial amount based on the signal and the patient information.

7. The arrangement of claim 6, wherein the instructions stored in the memory device are executable by the processor to cause the processor to compare the measured blood oxygen saturation level of the patient following delivery of supplemental oxygen to the patient at the initial rate or the initial amount to a reference pulse oximetric percentage, and to issue a warning in response to a determination that the measured blood oxygen saturation level of the patient does not meet the reference pulse oximetric percentage.

8. The arrangement of claim 7, wherein the instructions stored in the memory device are executable by the processor to cause the processor to titrate supplemental oxygen for delivery to the patient at an increased rate or an increased amount in response to the determination that the measured blood oxygen saturation level of the patient does not meet the reference pulse oximetric percentage.

9. The arrangement of claim 8, wherein the instructions stored in the memory device are executable by the processor to cause the processor to compare the measured blood oxygen saturation level of the patient following delivery of supplemental oxygen to the patient at the increased rate or the increased amount to the reference pulse oximetric percentage, and to issue an alarm in response to a determination that the measured blood oxygen saturation level of the patient does not meet the reference pulse oximetric percentage.

10. The arrangement of claim 9, wherein the instructions stored in the memory device are executable by the processor to cause the processor to, in response to a determination that the measured blood oxygen saturation level of the patient meets the reference pulse oximetric percentage, continue delivery of supplemental oxygen to the patient at the increased rate or the increased amount or cease further delivery of supplemental oxygen to the patient.

11. An automated oxygen monitoring system to deliver supplemental oxygen to a patient, the system comprising:
a controller having a memory device and a processor coupled thereto,
wherein the memory device has instructions stored therein that are executable by the processor to cause the processor to receive a signal from a sensor indicative of a measured blood oxygen saturation level of the patient, to titrate supplemental oxygen supplied by a source of supplemental oxygen for delivery to the patient based on the signal, and to continuously monitor the blood oxygen saturation level of the patient based on the signal.

12. The automated oxygen monitoring system of claim 11, wherein the instructions stored in the memory device are executable by the processor to cause the processor to titrate supplemental oxygen for delivery to the patient at an initial rate or an initial amount based on the signal.

13. The automated oxygen monitoring system of claim 12, wherein the instructions stored in the memory device are executable by the processor to cause the processor to compare the measured blood oxygen saturation level of the patient following delivery of supplemental oxygen to the patient at the initial rate or the initial amount to a reference pulse oximetric percentage, and to issue a warning in response to a determination that the measured blood oxygen saturation level of the patient does not meet the reference pulse oximetric percentage.

14. The automated oxygen monitoring system of claim 13, wherein the instructions stored in the memory device are executable by the processor to cause the processor to titrate supplemental oxygen for delivery to the patient at an increased rate or an increased amount in response to the determination that the measured blood oxygen saturation level of the patient does not meet the reference pulse oximetric percentage.

15. The automated oxygen monitoring system of claim 14, wherein the instructions stored in the memory device are executable by the processor to cause the processor to compare the measured blood oxygen saturation level of the patient following delivery of supplemental oxygen to the patient at the increased rate or the increased amount to the reference pulse oximetric percentage, and to issue an alarm in response to a determination that the measured blood oxygen saturation level of the patient does not meet the reference pulse oximetric percentage.

16. A method of delivering supplemental oxygen to a patient, the method comprising:
receiving, by a controller of an automated oxygen monitoring system, a signal provided by a sensor coupled to the oxygen monitoring system that is indicative of a measured blood oxygen saturation level of the patient;
titrating, by the controller, supplemental oxygen supplied by a source of supplemental oxygen for delivery to the patient based on the signal; and
continuously monitoring, by the controller, the measured blood oxygen saturation level of the patient based on the signal.

17. The method of claim 16, further comprising titrating, by the controller,
supplemental oxygen for delivery to the patient at an initial rate or an initial amount based on the signal.

18. The method of claim 17, further comprising:
comparing, by the controller, the measured blood oxygen saturation level of the patient following delivery of supplemental oxygen to the patient at the initial rate or the initial amount to a reference pulse oximetric percentage; and
issuing, by the controller, a warning in response to a determination that the measured blood oxygen saturation level of the patient does not meet the reference pulse oximetric percentage.

19. The method of claim 18, further comprising titrating, by the controller, supplemental oxygen for delivery to the patient at an increased rate or an increased amount in response to the determination that the measured blood oxygen saturation level of the patient does not meet the reference pulse oximetric percentage.

20. The method of claim 19, further comprising:
comparing, by the controller, the measured blood oxygen saturation level of the patient following delivery of supplemental oxygen to the patient at the increased rate or the increased amount to the reference pulse oximetric percentage; and
issuing, by the controller, an alarm in response to a determination that the measured blood oxygen saturation level of the patient does not meet the reference pulse oximetric percentage.
